(19) United States
(12) Patent Application Publication
(10) Pub. No.: US 2026/0345490 A1
(43) Pub. Date: Nov. 5, 2026

(54) LECITHIN-MODIFIED NANOSCALE OXYGEN CARRIERS (LENOX)

(71) Applicant: Universität Duisburg-Essen, Essen (DE)

(72) Inventors: Andrea Steinbicker, Oberursel (DE); Katja Ferenz, Essen (DE); Fabian Nocke, Essen (DE)

(21) Appl. No.: 19/108,068

(22) PCT Filed: Aug. 29, 2023
(86) PCT No.: PCT/EP2023/073579

§ 371 Date: Feb. 28, 2025

(30) Foreign Application Priority Data
Aug. 31, 2022 (EP) 22193221.3

(51) Int. Cl. A61K 9/107; A61K 31/025; A61K 47/24; A61K 47/42; B82Y 5/00

(52) U.S. Cl. CPC A61K 9/107; A61K 31/025; A61K 47/24; A61K 47/42; B82Y 5/00

(57) ABSTRACT

The present invention relates to a novel composition for the development of stable artificial oxygen carriers based on perfluorocarbons, their preparation and their use as synthetic blood and oxygen replacement carriers and volume substitutes.

The present invention relates to new, stable artificial oxygen carriers based on perfluorocarbons, their production and their use as synthetic blood substitutes.

### BACKGROUND TO THE INVENTION

Perfluorocarbons (PFCs) are synthetically produced, perfluorinated carbon compounds (hydrocarbons whose hydrogen atoms are completely replaced by halogens, usually fluorine atoms). Due to cavities between the individual molecules, they have a high solubility for respiratory gases such as oxygen and carbon dioxide compared to water, depending on the partial pressure, and can therefore take over or support oxygen transport in the blood instead of or together with erythrocytes. Oxygen uptake and release is two times faster than with erythrocytes and directly proportional to the oxygen partial pressure. More than 90% of the dissolved oxygen is released to the tissue, which is three times the oxygen extraction rate (oxygen release to the tissue) of an erythrocyte. Due to the very high carbon-fluorine binding energy, perfluorocarbons are chemically and metabolically inert, so that they do not react even with highly reactive compounds and do not form toxic degradation products. In terms of degradation, the perfluorodecalin (PFD) we use will be excreted in the breath due to its high vapor pressure. High vapor pressure means that a liquid evaporates under mild conditions. In this case, this means that PFD reaches the lungs as a liquid and is then vaporized and exhaled.

Due to the insolubility of perfluorocarbons in both water and oil, either emulsification or encapsulation is required in order to use corresponding PFC-based oxygen carriers (PFOCs) as an additive to a perfusion medium or transfusion medium.

EP0282948B1 and EP0282949B1 describe aqueous emulsions of perfluorocarbons in which phospholipids, among other things, are used as emulsifiers. Additional emulsifier additives may also be present, such as albumin, in particular bovine serum albumin (BSA). This can be present in the emulsion in an amount of 0.2-2% by weight as an “oncontic agent”.

CN111214459A (“Perfluorocarbon and albumin nanoparticles and application in production of tumor treating medicine thereof”) describes nanoparticles of perfluorocarbon and albumin as a drug delivery system for “phosphonic acid drugs”. A production process is described in which the “phosphonic acid drug” is added to the perfluorocarbon/albumin nanoparticles in an emulsion, which may contain lecithin and cholesterol, among other things, in order to produce biologically active double-membrane particles. This publication therefore concerns liposomes. Nanoparticles with a shell of albumin and lecithin are not described. The application is not related to artificial oxygen carriers or blood replacement solutions, but to tumor therapy.

CN109908085A describes similar emulsions of BSA, lecithin and perfluorocarbons. The BSA is not used as an emulsifier, but as an example protein for the drug carrier system. However, nanoparticles with a shell of albumin and lecithin are not explicitly described in the application. The application is also not related to artificial oxygen carriers or blood replacement solutions, but to the treatment of heart failure.

EP0033402A1 describes a perfusate liquid which consists of a previously used crystalloid volume replacement solution (Ringer's solution) in which albumin is dissolved and which may additionally contain, among other things, a perfluorocarbon and an emulsifying agent. The emulsifying agent can be a phospholipid, for example. In this case, the albumin is not used as an emulsifier, but forms an additive in the carrier solution.

Jacoby C, et al. (in: Probing different perfluorocarbons for in vivo inflammation imaging by 19F MRI: image reconstruction, biological half-lives and sensitivity. NMR Biomed. 2014 March; 27(3):261-71. doi: 10.1002/nbm.3059. Epub 2013 Dec. 19. PMID: 24353148) describe PFCE emulsions generated by high-pressure homogenization and further containing 10% w/w crown ether and 4% w/w purified egg lecithin E 80 S (Lipoid GmbH, Ludwigshafen, Germany) in isotonic buffer.

U.S. Pat. No. 4,866,096 A describes a stable aqueous emulsion containing 10-59% of an oxygen-transferring, saturated perfluorodecalin, triglycerides of fatty acids and 0.5-7% of a surface-active phospholipid and optionally albumin. The emulsion is prepared under cooling in a microfluidizer. It is further described that the emulsion is used as a synthetic blood substitute.

Jagers et al. (in: “Perfluorocarbon-based oxygen carriers: from physics to physiology”, PFLUGERS ARCHIV—EUROPEAN JOURNAL OF PHYSIOLOGY, Vol. 473, No. 2, Nov. 3, 2020, pages 139-150) describe stable emulsions containing perfluorodecalin (PFD) together with lecithin. Albumin is specifically mentioned in this publication as an emulsifier to increase the stability of the emulsion. However, a combination of albumin, lecithin and perfluorodecalin is not described.

U.S. Pat. No. 4,186,253 A describes the non-cooled production of a perfluorocarbon emulsion using nozzle-type emulsifiers, whereby the emulsion is to be used in organ transplantation. The emulsion also contains a modified Ringer's solution in which albumin is dissolved.

WO 94/18954 A1 describes microemulsions for use as blood substitutes containing albumin, lecithin and perfluorodecalin.

In the in vivo research approaches to date, perfluorocarbons are only used as artificial oxygen carriers in the form of emulsions, whereby the instability of the emulsions or the short half-life, among other things, still cause difficulties (Lambert, E., Janjic, J. M. Quality by design approach identifies critical parameters driving oxygen delivery performance in vitro for perfluorocarbon based artificial oxygen carriers. Sci Rep 11, 5569 (2021). https://doi.org/10.1038/s41598-021-84076-1).

Although products such as Fluosol-DA®, Perftoran® and Oxycyte® have been tested in clinical trials, none of the preparations have yet been approved in Europe or the USA due to side effects (Ferenz K B, Steinbicker A U. Artificial Oxygen Carriers-Past, Present, and Future—a Review of the Most Innovative and Clinically Relevant Concepts. J Pharmacol Exp Ther. 2019 May; 369(2):300-310. doi: 10.1124/jpet.118.254664. Epub 2019 Mar. 5. PMID: 30837280).

Newer preparations, e.g. Oxygent® (a 60% PFC emulsion with 58% perfluorooctyl bromide, 2% perfluorodecyl bromide and egg yolk phospholipids), work with a combination of the somewhat less stabilizing (but better tolerated) phospholipids, e.g. from egg yolk and high molecular weight PFCs such as perfluorodecyl bromide (CF₃(CF₂)₁₀Br), perfluorotributylamine (Ne.g. from egg yolk and high-molecular PFCs such as perfluorodecyl bromide (CF₃(CF₂)₁₀Br), perfluorotributylamine (N(CF₂CF₂CF₂CF₃)₃) or perfluoromethylcyclohexylpiperidine (C₁₂F₂₂N) (Ferenz K B, 2015. Artificial oxygen carriers—how much longer do we have to wait?Hemotherapy 25, 27-36).

Despite certain advances in this field, there is still the problem that perfluorocarbons cannot be emulsified in aqueous solution in such a stable manner that they can be effectively used as an additive to a perfusion medium or transfusion medium. It is thus the task of the present invention to provide correspondingly improved processes for the production of artificial oxygen carriers based on perfluorocarbons and correspondingly improved compositions. Further tasks of artificial oxygen carriers are presented to the person skilled in the art when reading the “state of the art” in the following in a detailed description and in figures and examples.

In a first aspect of the present invention, the above task is solved by a method for the preparation of artificial oxygen carriers based on perfluorocarbons (PFOCs). The method comprises the steps of a) providing a suitable aqueous albumin solution and mixing it with at least one suitable artificial oxygen carrier based on perfluorocarbons, b) suitably adding lecithin, c) pre-emulsifying by rapid stirring under suitable cooling, d) emulsifying the pre-emulsion from step c) under pressure via a high-pressure homogenizer under suitable cooling and then storing it for at least 30 min, also under suitable cooling.

Preferred is a method according to the present invention, wherein albumin in a concentration between 2-20%, preferably 5-10%, more preferably 5%, perfluorocarbon perfluorodecalin (PFD) in a concentration between 10-50%, preferably 17%, lecithin in a concentration between 1-16%, preferably of, in a medium selected from water and electrolytes in a plasma-like composition, preferably Sterofundin® ISO, Ringerfundin®, Ringer solution and hydroxy-ethyl starch, STEEN solution, OCS solution and other crystalloid and colloidal volume replacement solutions.

A method according to the present invention is further preferred, wherein the artificial oxygen carrier is produced exclusively from the specified materials.

In a second aspect of the present invention, the above problem is solved by an artificial oxygen carrier prepared by a method according to the present invention.

The artificial oxygen carrier according to the invention has a higher stability compared to non-lecithin-containing artificial oxygen carriers based on perfluorocarbons (PFOCs) and can be produced directly in the typical crystalloid and colloidal volume replacement solutions, such as Sterofundin® ISO.

In a third aspect of the present invention, the above problem is solved by using the artificial oxygen carrier according to the present invention as a perfusion or transfusion solution or as a synthetic blood substitute.

The present inventors have already developed artificial oxygen carriers based on perfluorocarbons, which were emulsified with 5% albumin (albumin-derived perfluorocarbon-based artificial oxygen carrier, A-AOC) and which have already shown promising results (see, among others, Wrobeln A, et al. Albumin-derived perfluorocarbon-based artificial oxygen carriers: A physico-chemical characterization and first in vivo evaluation of biocompatibility. Eur J Pharm Biopharm. 2017 June; 115:52-64. https://doi.org/10.1016/j.ejpb.2017.02.015. Epub 2017 Feb. 20. PMID: 28232105).

So far, these oxygen carriers have been successfully used for tissue oxygenation in the blood exchange model (in the rat) and for the prevention of diver's disease (in the rat) (Wrobeln A, Jagers J, Quinting T, Schreiber T, Kirsch M, Fandrey J, Ferenz K B. Albumin-derived perfluorocarbon-based artificial oxygen carriers can avoid hypoxic tissue damage in massive hemodilution. Sci Rep. 2020 Jul. 20; 10(1):11950. doi: 10.1038/s41598-020-68701-z. PMID: 32686717; PMCID: PMC7371727, Mayer D, Guerrero F, Goanvec C, Hetzel L, Linders J, Ljubkovic M, Kreczy A, Mayer C, Kirsch M, Ferenz K B. Prevention of Decompression Sickness by Novel Artificial Oxygen Carriers. Med Sci Sports Exerc. 2020 October; 52(10):2127-2135. Doi: 10.1249/MSS.0000000000002354. PMID: 32251255.). The inventors were also able to demonstrate successful tissue oxygenation in the field of transplantation medicine using extracorporeally perfused organs (organs outside the organism, such as isolated heart or isolated kidney) (Jagers J, Wrobeln A, Ferenz K B. Perfluorocarbon-based oxygen carriers: from physics to physiology. Pflugers Arch. 2021 February; 473(2):139-150. Doi: 10.1007/s00424-020-02482-2. Epub 2020 Nov. 3. PMID: 33141239; PMCID: PMC7607370).

DE102008045152A1 and EP 2 296 635 B1 relate to artificial oxygen carriers and their use. However, the oxygen carriers are not yet stable in every carrier solution that is clinically desirable. In particular, no stable emulsions could be generated in the typical crystalloid and colloidal volume replacement solutions, such as Sterofundin® ISO, meaning that the oxygen carriers cannot be used universally.

In a first aspect of the present invention, as mentioned above, the above task is solved by a method for the production of artificial oxygen carriers based on perfluorocarbons (PFOCs). The method first comprises the step of providing a suitable aqueous albumin solution.

In principle, all aqueous carriers suitable for use as artificial oxygen carriers based on perfluorocarbons can be used. Preferably, the albumin is dissolved in a suitable aqueous buffer, water, a balanced full electrolyte solution for infusion therapy, such as Sterofundin® ISO, Ringerfundin®, Ringer solution, STEEN solution, OCS solution and/or in hydroxyethyl starch. The typical crystalloid and colloidal volume replacement solutions, such as Sterofundin® ISO, are particularly suitable.

In principle, the albumin used is not limited to one species. Preferred is a method according to the present invention, wherein the albumin is selected from the group consisting of mammalian albumin, human serum albumin (HSA) and bovine serum albumin (BSA), or suitable derivatives thereof, such as pegylated albumins.

The albumin solution is then mixed with at least one suitable artificial oxygen carrier based on perfluorocarbons. Any artificial oxygen carrier based on perfluorocarbons can be used. Preferred is a method according to the present invention, wherein the artificial oxygen carrier based on perfluorocarbons is selected from the group consisting of perfluorodecalin, perfluorodecylbromide, perfluorotributylamine, perfluoromethylcyclohexylpiperidine, perfluorodichlorooctane, perfluorotertbutylcyclohexane, perfluoro-15-crown-5-ether, dodecafluoropentane, perfluorooctylbromide and perfluorocyclohexane. Also suitable are artificial oxygen carriers based on perfluorocarbons as published in Wesseler (Eugene P. Wesseler, Ronald Iltis, Leland C. Clark, The solubility of oxygen in highly fluorinated liquids, Journal of Fluorine Chemistry, Volume 9, Issue 2, 1977, Pages 137-146, ISSN 0022-1139, https://doi.org/10.1016/S0022-1139(00)82152-1), in particular in Table 2.

Lecithin is then added in a suitable manner. Preferred is fat-free vegetable lecithin, for example from soy with a purity of ≥97%. These new, stable oxygen carriers, which the inventors refer to as “lecithin modified nanoscale oxygen carriers” (LENOX), differ from the previous oxygen carriers, the precursors called albumin-derived artificial oxygen carriers (A-AOCs) and organ-life fluid (OLF), in that lecithin is now added during synthesis. In addition to a higher operating pressure of the manufacturing machine and a multi-cyclic synthesis, the phospholipid lecithin was also incorporated into the albumin shell. These changes made it possible to completely eliminate various stability problems of the previously known emulsions. Lecithin is a mixture of phosphatidylcholine and other phospholipids and a component of animal and plant cell membranes.

The present emulsion has the advantage of not requiring any further stabilizers in addition to the two emulsifiers albumin and lecithin. A method according to the present invention is therefore preferred, wherein the artificial oxygen carrier is produced or consists exclusively of the specified materials. Both albumin and lecithin are already clinically approved for intravenous use.

Usually, the components used to produce an emulsion are first premixed to form a coarsely dispersed pre-emulsion, which can also be referred to as a raw or pre-emulsion or premix. Homogenization then takes place, whereby the disperse phase is broken down into droplets (fine emulsification). The droplet size spectrum of the raw or pre-emulsion shifts significantly towards smaller droplets. O/W emulsions for parenteral application are usually prepared by first premixing an oil phase and water phase to form a pre-emulsion using a rotor-stator stirrer.

Thus, according to the invention, the subsequent pre-emulsification of the mixture produced above (which is usually still separated into phases) is also carried out by rapid stirring with suitable cooling. A preferred process according to the present invention is one in which the pre-emulsification is carried out by rapid stirring with a homogenizer at between 8,000 and 12,000 rpm, such as with an Ultra-Turrax® at 9,500 rpm.

As a final step, the pre-emulsion is emulsified as above under (high) pressure via a high-pressure homogenizer, e.g. a counterjet disperser (see e.g. DE 10 2018 205 493 A1) under suitable cooling and subsequent storage for at least 30 min, also under suitable cooling. A method according to the present invention is preferred, wherein the emulsification of the pre-emulsion takes place at a pressure between 10,000 PSI (approx. 689 bar) and 40,000 PSI (approx. 2758 bar), preferably at 30,000 PSI (2068 bar) and is repeated several times if necessary, preferably between 1 and 12 times (further preferably between 5 and 10, optimally repeat 8 times).

The steps of the process, in particular homogenization and emulsification, are carried out under suitable cooling at <10 °C or lower, preferably at 4 °C. Of course, freezing should be avoided.

The inventors have now succeeded for the first time in synthesizing artificial oxygen carriers based on perfluorocarbons (PFOCs), which have a high stability with only the three components albumin, lecithin and PFD without further additives (such as triblock copolymers like Pluronic) and can also be produced directly in a clinically approved crystalloid or colloidal volume replacement solution (preferably Sterofundin® ISO) and other carrier solutions, which was not possible before.

A method according to the present invention is particularly preferred, wherein albumin is emulsified in water in a concentration of between 2-20%, preferably of 5-10%, more preferably of about 5%, perfluorodecalin (PFD) in a concentration of between about 10-50%, preferably of about 17%, lecithin in a concentration of between about 1-16%, preferably of about 2%.

The proportion of albumin used leads to a physiological colloid osmotic pressure as an important advantage, whereby surprisingly an optimum was found at around 5%. The combination of PFOC+albumin+lecithin therefore leads to a stable formulation and a ready-to-use product in a full electrolyte/volume replacement solution. However, artificial oxygen carriers (AOC) with other albumin contents (between 2-20%, 4-10%, 4-6%) are also advantageous for a stable formulation.

In the context of the present invention, “about” means a deviation of ±10%, unless otherwise specified.

A method according to the present invention is particularly preferred, wherein the emulsion is prepared at 20,000 PSI with BSA in water and the particle diameter in the emulsion is between 50 nm and 400 nm, further preferably between 60 nm and 300 nm, optimally between 70 nm and 250 nm. See in particular synthesis 5, below.

A method according to the present invention is particularly preferred, wherein the mean particle diameter in the emulsion prepared with BSA is 92.5 nm±8.5 nm and the oxygen release is 3.99 μmol/mL±0.20 μmol/mL.

A method according to the present invention is particularly preferred, wherein the artificial oxygen carrier is produced exclusively from the indicated materials, or albumin and lecithin are present as the only carriers and/or emulsifiers.

Another aspect of the present invention relates to an artificial oxygen carrier prepared by a method according to the present invention.

Preferred is an artificial oxygen carrier according to the present invention comprising about 5% albumin, perfluorodecalin (PFD) at a concentration of about 17% and lecithin at a concentration of about 2% in a medium selected from water, Sterofundin® ISO, Ringerfundin®, STEEN-Solution, OCS solution, Ringer's solution and hydroxy ethyl starch.

The artificial oxygen carrier according to the present invention is characterized by the fact that it has a higher stability compared to non-lecithin-containing artificial oxygen carriers based on perfluorocarbons (PFOCs). This can be determined in particular by measuring the viscosity particle size and particle distribution of the artificial oxygen carrier (see examples).

Another aspect of the present invention relates to a synthetic blood substitute comprising the artificial oxygen carrier according to the present invention.

A further aspect of the present invention relates to the use of the artificial oxygen carrier according to the present invention as a perfusion or transfusion solution or as a synthetic blood substitute.

Another aspect of the present invention relates to the artificial oxygen carrier according to the present invention for use in the treatment and prevention of oxygen deficiency and/or blood deficiency in an organ or patient, in particular in a human.

A further aspect of the present invention relates to a method for treating and/or preventing oxygen deficiency and/or blood deficiency in an organ or patient, comprising using the artificial oxygen carrier according to the present invention as a perfusion or transfusion solution or as a synthetic blood substitute, in particular in a human.

The composition of the oxygen carrier according to the invention (LENOX) preferably consists of albumin, perfluorodecalin (PFD) and lecithin in water, wherein the concentration of albumin is between 2-20%, preferably 5-10%, more preferably about 5%, the PFD concentration is between 10-50% (preferably 17%) and the lecithin concentration is between 1-16% (preferably 2%). The mean particle diameter with BSA is 92.5 nm±8.5 nm and the oxygen release is 3.99 μmol/mL±0.20 μmol/mL.

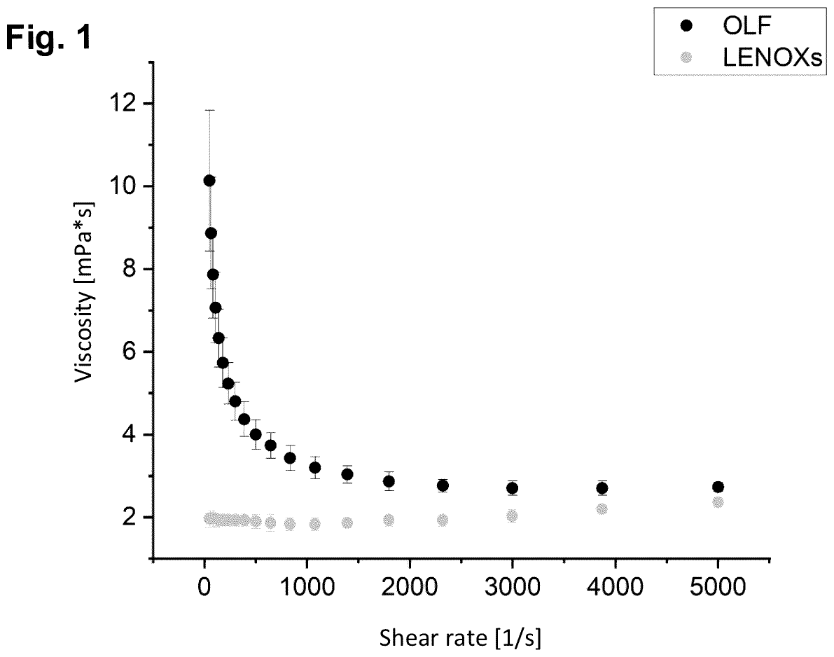

Surprisingly, the addition of lecithin also leads to significantly improved physicochemical properties of the nanoparticles obtained in this way compared to the previous OLF particles based solely on albumin. The oxygen carriers according to the invention (LENOX) are significantly smaller, less polydisperse and less cloudy than the previous OLF oxygen carriers (FIG. 1). They can be synthesized in particular with HSA as well as BSA and are stable in a wider temperature range (−20 to ±100 °C).

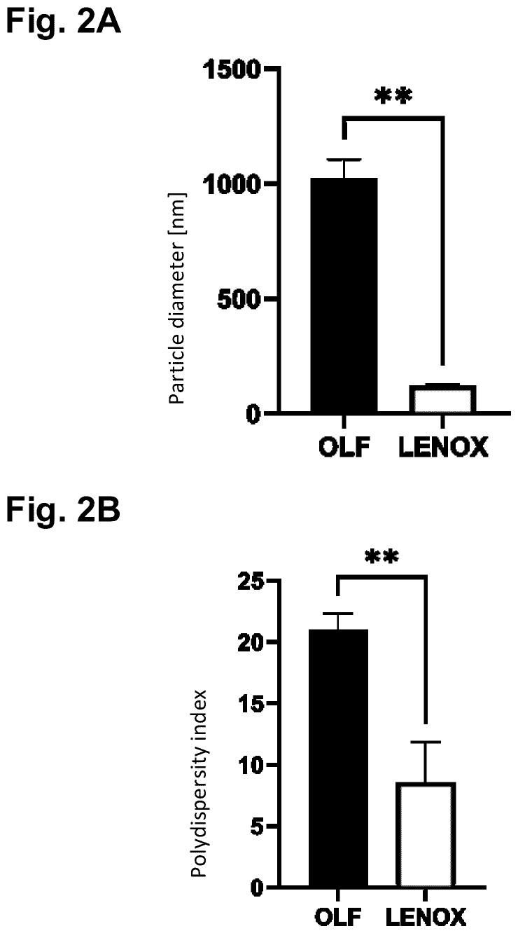

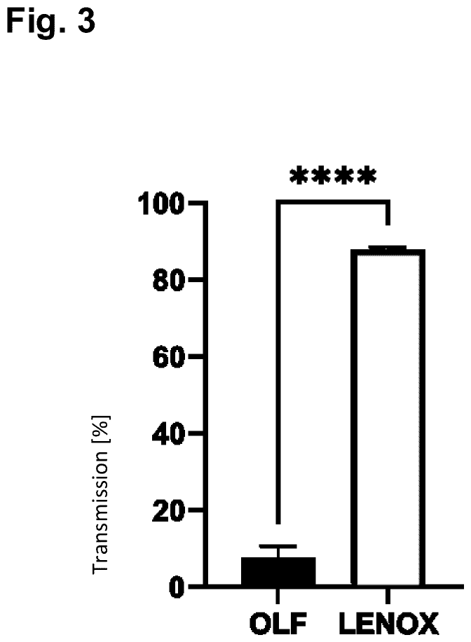

In addition, they have a significantly lower viscosity than the OLF particles (FIG. 2). In particular, however, the oxygen carriers according to the invention are compatible with Ringer solution, Sterofundin® ISO (FIG. 3), Ringerfundin®, STEEN solution, OCS solution and 6% HES (130/0.4) in the long term and can be synthesized directly and stably in these solutions. Until now, for example, it has not been possible to synthesize stable oxygen carriers without additional additives in approved volume replacement solutions such as Sterofundin® ISO.

The oxygen carriers according to the invention have very good properties in terms of stability, viscosity, particle size and oxygen release.

The present invention will now be further described in the following examples with reference to the accompanying figures, but without being limited thereto. For purposes of the invention, all cited documents and publications are hereby incorporated by reference in their entirety. Shown in the figures:

FIG. 1: Viscosity curve of the OLF particles compared to the LENOX (example synthesis I, HSA).

FIG. 2: Diameter (A) and polydispersion index (B) of the OLF particles compared to the LENOX (example synthesis I, HSA).

FIG. 3: Transmission of OLF particles and LENOX (example synthesis I, HSA).

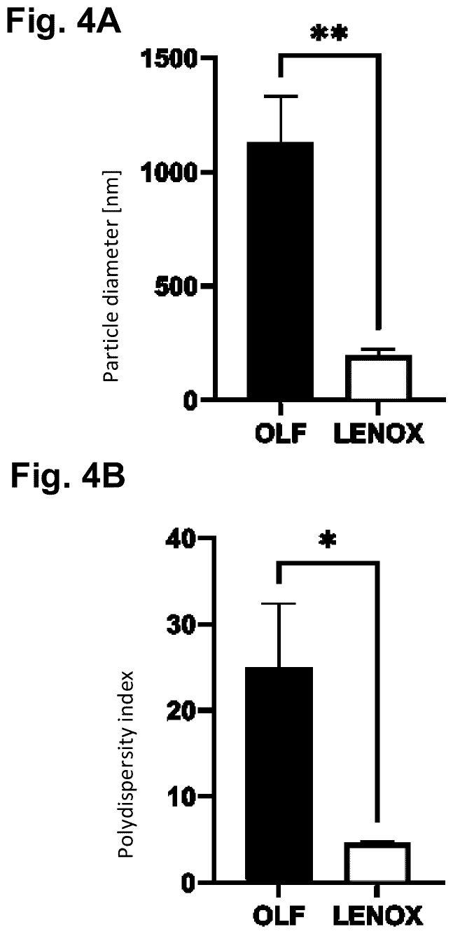

FIG. 4: Diameter and polydispersion index of OLF particles and LENOX synthesized in Sterofundin® ISO (example synthesis II).

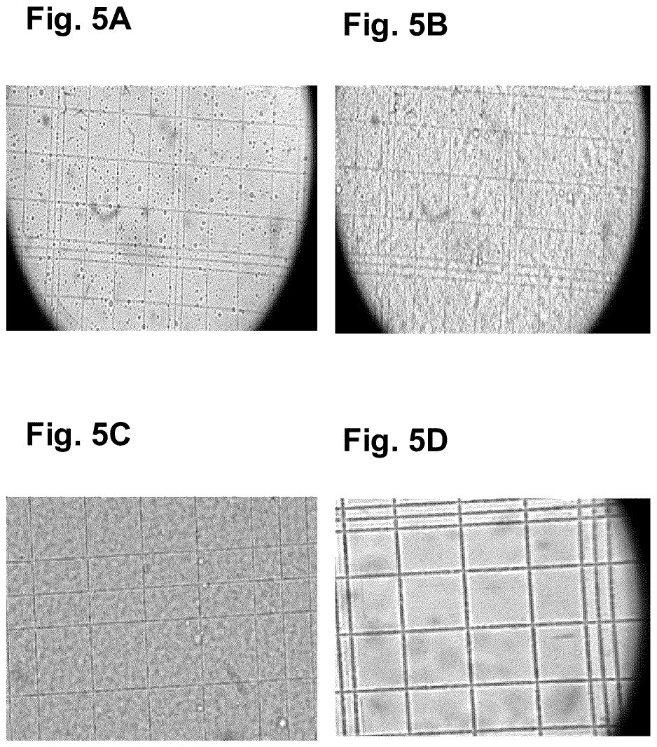

FIG. 5: Microscopy images of OLF particles (state of the art) synthesized in Ringer's solution (A), HES 6% (B) and Sterofundin® ISO (C). The noisy background in images B and C indicates that these emulsions have been destroyed. The emulsion in image A is still intact, but shows particles>1 μm. The LENOX (example synthesis I, BSA) are not visible under the microscope as they are below the resolution limit (D). The artifacts in image D are caused by residues on the Neubauer chamber and do not originate from the emulsion droplets.

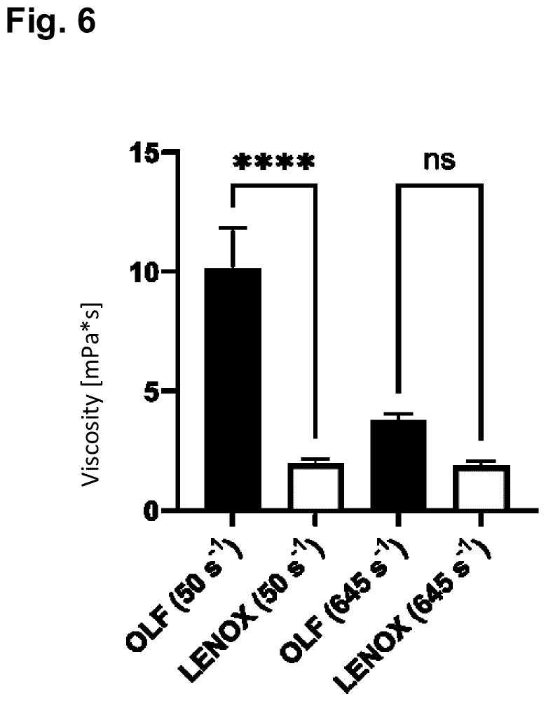

FIG. 6: Viscosity of the OLF particles compared to the LENOX, synthesized in 6% HES (130/0.4) (example synthesis IV).

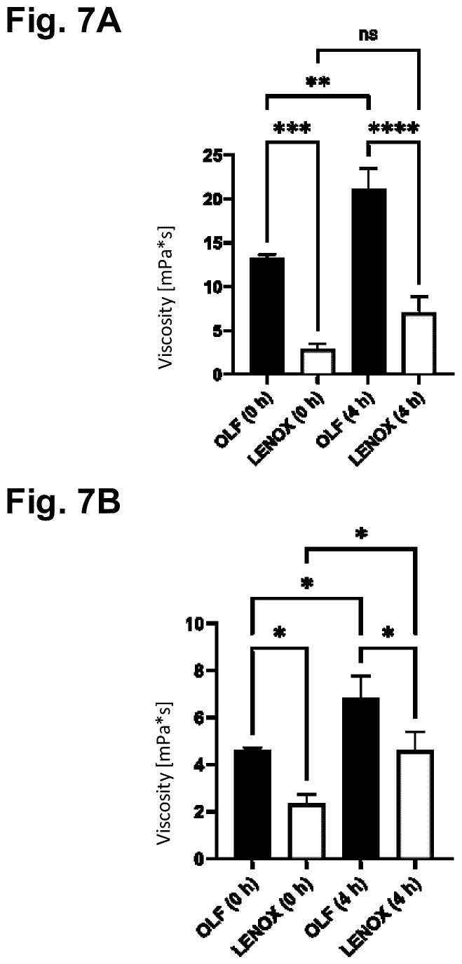

FIG. 7: Viscosity of OLF particles and LENOX (example synthesis II) before and after storage for 4 h at 37 °C.

### EXAMPLES

### List of Abbreviations

| | |
|---|---|
| Lecithin-modified nano-oxygen carrier | LENOX |
| (Lecithin-modified nano oxygen carrier) | |
| Perfluorodecalin | PFD |
| AOC in organ life fluid | OLF |
| Hydroxyethyl starch | HES |
| University of Duisburg-Essen | UDE |
| bovine serum albumin | BSA |
| human serum albumin | HSA |
| Perfluorocarbon-based artificial oxygen carriers | PFOCs |
| (Perfluorocarbon-based oxygen carrier) | |
| Albumin-based artificial oxygen carriers | A-AOCs |
| (Albumin-based artificial oxygen carrier) | |
| Artificial oxygen carriers | AOC |
| (Artificial oxygen carrier) | |
| 10th/50th/90th percentile | d10/d50/d90 |
| Range of particle size distribution | S |

### Materials and Equipment

For the syntheses of the artificial oxygen carriers, perfluorodecalin HP (F2 Chemicals, Lancashire, UK), Millipore water (Q-Pod, Merck, Darmstadt, DE) and either albumin fraction V (bovine serum albumin, Roth, Karlsruhe, DE) or Albunorm (human serum albumin, Octapharma, Langenfeld, DE) were homogenized with a high-pressure homogenizer (LM20, Microfluidics, Waterloo, CAN) after a pre-emulsion had been prepared with an Ultraturrax (IKA).

Lecithin (97%, Roth, Karlsruhe, DE) was also used for the newly developed oxygen carrier (LENOX).

Sterofundin® ISO (B. Braun, Melsungen, DE), HES 6% (Fresenius Kabi, Bad Homburg, DE) and Ringer's solution (Fresenius Kabi, Bad Homburg, DE) were used as exemplary saline solutions.

The particle size and the polydispersion index were determined using dynamic light scattering (Stabino Nano-flex, Particle Metrix, Inning am Ammersee, DE). The oxygen capacity was measured with a respirometer (O2k, Oroboros Instruments, Innsbruck, AUT) and the turbidity with a photometer (Specord 5600, Analytikjena, Jena, DE). Viscosity was determined using a rheometer (MCR 92, Anton Paar, Ostfildern, DE). The viscosity was determined at two different shear rates, as some emulsions exhibited non-Newtonian behavior. A very low shear rate (50/s) and a physiological shear rate (645/s) were selected.

A light microscope (AXIO Lab.A1, Zeiss, Oberkochen, DE) was used to visualize the microparticles.

### Synthesis Process

The synthesis of the pure Organ Life Fluid particles (OLF particles) corresponded to that described in patent application DE102021211272.2. 20 mL of albumin (BSA or HSA) and 4 mL of PFD were added and pre-emulsified with an Ultra-Turrax (T25 Basic, IKA-Werke, Staufen, DE) at 9,500 rpm. The pre-emulsion was then added to the high-pressure homogenizer and the entire volume was processed once at a pressure of 20,000 PSI (approx. 1379 bar).

During the process, the outlet coil of the appliance and therefore also the product must be cooled with ice. After homogenization, the product was stored in a container filled with ice for at least 30 minutes.

Synthesis I-IV is carried out at a pressure of 30,000 PSI and a cycle rate of eight times, from synthesis V the pressure changes to 20,000 PSI and the number of cycles changes to seven times.

### Synthesis I

For the synthesis of LENOX, 20 mL of a 5% albumin solution (bovine serum albumin (BSA) or human serum albumin (HSA)) and 4 mL PFD were added. The 2-phase mixture was mixed with 0.4 g lecithin and pre-emulsified with an Ultra-Turrax (9,500 rpm). The pre-emulsion was then added to the high-pressure homogenizer and processed eight times at a pressure of 30,000 PSI (approx. 2068 bar). During the process, the outlet coil of the device and therefore also the product must be cooled with ice. After homogenization, the product is stored in a container filled with ice for at least 30 minutes.

### Synthesis II

For the synthesis of LENOX in Sterofundin® ISO, 1 g of bovine serum albumin was dissolved in 20 mL of Sterofundin® ISO. First 4 mL PFD and then 0.4 g lecithin were added to this solution. The mixture was pre-emulsified with an Ultra-Turrax at 9,500 rpm. The pre-emulsion was then added to the high-pressure homogenizer and the entire volume was processed eight times at a pressure of 30,000 PSI (approx. 2068 bar). During the process, the outlet coil of the device and therefore also the product had to be cooled with ice. After homogenization, the product was stored in a container filled with ice for at least 30 minutes.

### Synthesis III

For the synthesis of LENOX in Ringer's solution, 1 g of bovine serum albumin (BSA) was dissolved in 20 mL of Ringer's solution. First 4 mL PFD and then 0.4 g lecithin were added to this solution. The mixture was pre-emulsified with an Ultra-Turrax at 9,500 rpm. The pre-emulsion was then added to the high-pressure homogenizer, and at a pressure of 30,000 PSI (approx. 2068 bar), the entire volume was cycled through the high-pressure homogenizer eight times. During the process, the outlet coil of the device and therefore also the product must be cooled with ice. After homogenization, the product was stored in a container filled with ice for at least 30 minutes.

### Synthesis IV

For the synthesis of LENOXs in hydroxyethyl starch (HES, 6%), 1 g of bovine serum albumin (BSA) was dissolved in 20 mL of HES (6%). First 4 mL PFD and then 0.4 g lecithin were added to this solution. The mixture was pre-emulsified with an Ultra-Turrax at 9,500 rpm. The pre-emulsion was then added to the high-pressure homogenizer and the entire volume was processed eight times at a pressure of 30,000 PSI (approx. 2068 bar). During the process, the outlet coil of the device and therefore also the product must be cooled with ice. After homogenization, the product was stored in a container filled with ice for at least 30 minutes.

### Statistics:

OriginPro® software (version 2020) was used for the statistical evaluation of the analysis data.

The test data shown below are the mean values of the groups±standard deviation. The standard deviation is shown in the form of error bars. In order to determine the statistical significance between two data sets, the data sets were first examined for normal distribution using the Shapiro-Wilk test. As all data sets were normally distributed, unpaired t-tests or one-way ANOVA tests with subsequent post hoc analysis were performed. Two data sets are considered significantly different if the mean value of the population differs from the test difference at an error level of *p<0.05. If there is a significant difference, the two data sets are marked as follows: p≤0.05 (*), p≤0.01 (**), p≤0.001 (***) and p≤0.0001 (****). If the error level *p>0.05, the two data sets are not significantly different (ns). All data sets consist of a sample size of n=3 samples each.

### Results

The LENOX have an average particle diameter of 92.5±8.5 nm (Synthesis I, BSA) or 123.5±2.2 nm (Synthesis I, HSA) and release 3.99±0.2 μmol oxygen per mL sample (at 17 vol. %). The emulsion has the properties of a Newtonian fluid, whereby the viscosity changes only minimally with increasing shear rate. Compared to the LENOXs, the OLF particles form a non-Newtonian fluid, and the viscosity changes significantly with changing shear rate (see FIG. 1).

The mean particle diameter and the polydispersion index are significantly different between the OLF particles (HSA) and the LENOXs. The average particle diameter of the measured samples was 1020.8±85.9 nm for the OLF particles and 123.5±2.2 nm for the LENOX (Synthesis I, HSA) (*p=0.003). With a polydispersion index of 20.99±1.33, the emulsion of the OLF particles is significantly (*p=0.031) more polydisperse than the emulsion of the LENOX (synthesis I, HSA) with a polydispersion index of 8.58±3.28.

An optically visible difference between the OLF emulsion and the LENOX emulsion is the turbidity. By measuring the transmission of both emulsions, it could be shown that the LENOX emulsion (Synthesis I, HSA) is significantly (*p<0.001) more transparent to light with a wavelength of 860 nm than the OLF emulsion. Thus, the OLF emulsion appears very cloudy, whereas the LENOXs emulsion appears clear.

In order to investigate the applicability in a perfusion medium, both the OLF particles and the LENOX were synthesized in different perfusion media (Ringer's solution, Sterofundin® ISO and HAES 6%). Significant differences in the mean particle diameter (*p=0.019) and polydispersion index (*p=0.001) were observed when synthesized in Sterofundin® ISO. These differences are comparable to the differences in FIG. 2.

When synthesized with Ringer's solution, an intact emulsion was formed with both the OLF particles and the LENOX. However, the OLF particles were visible under an optical microscope, whereas the LENOX particles were too small to be seen under an optical microscope. The OLF particles are therefore significantly larger than the LENOX particles in the same medium when synthesized in Ringer's solution. The light microscope images of the OLF particles in HES 6% and in Sterofundin® ISO show a strong background noise. This is a sign of a destroyed emulsion. Thus, no stable OLF emulsions are formed in HES 6% and in Sterofundin® ISO.

This behavior is also reflected in the viscosity. For example, the viscosity in HES 6% is significantly (*p=0.031 at 50/s and *p=0.037 at 650/s) higher for the OLF particles than for the LENOX (Synthesis IV). FIG. 6 shows the viscosities as a function of two different shear rates, since the OLF particles have a strong non-Newtonian character.

In order to investigate the influence of the temperature of a normothermic perfusion (37 °C) on the emulsions, samples of OLF particles and LENOX were stored in Sterofundin® ISO (Synthesis II) for 4 h at 37 °C and the viscosity was determined before and after storage. A significant (*p>0.001, *p=0.003, *p=0.007) difference was found between the two emulsions. The OLF particles have a significantly higher viscosity than the LENOX both before and after storage. Only after storage at a shear rate of 650/s are the two emulsions not significantly different from each other (*p=0.056).

### Summary

LENOX are significantly smaller, less polydisperse and less cloudy than OLF particles.

LENOX can be synthesized with both HSA and BSA.

LENOX are more stable at different temperatures (−20 to +100 °C).

LENOX are compatible with Ringer's solution, Sterofundin® ISO, STEEN solution, OCS solution and HAES 6%, OLF particles show incompatible behavior (viscosity, stability, etc.) in many of these solutions.

### Further Experiments

### Methods

### Dynamic Light Scattering

The particle size distribution was determined by dynamic light scattering (DLS) with Nano-flex (Microtrac). The refractive indices were taken from previous work. The high and low temperature viscosity was determined individually for each batch and each test. After a zero measurement for 300 s with water, 1 mL of the sample was filled into a 2 mL reaction vessel and measured three times for 300 s.

The percentiles d10, d50 and d90 were determined to analyze the data. In addition, the range (S) of the particle size distribution was calculated using equation (1).

$$S = \frac{d 90 - d 10}{d 50} \qquad ( 1 )$$

### Viscosity

A rheometer (MCR 92, Anton Paar) was used to measure the shear rate-dependent viscosity. The internal software calibration process was used to set up and calibrate the device. For the measurement, 1 mL of the sample was placed on the measuring table and the system was set to measuring mode. The system was heated to 20 °C and the viscosity was measured at different shear rates. Immediately after the first measurement, the system was heated to 37 °C and the measurement was repeated to determine the high-temperature viscosity.

### Oxygen Release

Oxygen release was measured using an SDR SensorDish Reader (PreSens) under hypoxic conditions. For this purpose, 0.5 ml of water or saline solution per well was prepared in a special 24-well plate equipped with oxygen sensors (OxoDish OD 24, PreSens). This plate was stored under a hypoxia workstation (whitley H35) (1% O₂, 5% CO₂, 94% N₂) until the oxygen content no longer changed. 2.5 mL of the sample was transferred to a gas-tight flask and oxygenated for 15 minutes with 100% O₂ at a gas flow rate of 0.5 mL/min. 0.5 mL of the previously oxygenated sample was added to each well and the oxygen release was measured until the value returned to baseline.

### Zeta Potential

The zeta potential was determined by measuring the scattering potential (Stabino Zeta, Microtrac). For the zeta potential measurements, 1 ml of the sample was filled into the specific measuring flask and the flask was connected to the measuring system (Stabino, Microtrac). The sample was measured ten times for 10 s each and the results were averaged.

### Further Syntheses

### Synthesis V

For the synthesis of LENOX, 20 mL of a 5% albumin solution (bovine serum albumin (BSA) or human serum albumin (HSA)) and 4 mL PFD were added. The 2-phase mixture was mixed with 0.4 g lecithin and pre-emulsified with an Ultra-Turrax (9,500 rpm). The pre-emulsion was then added to the high-pressure homogenizer and the entire volume was processed seven times at a pressure of 20,000 PSI (approx. 1379 bar). During the process, the outlet coil of the device and therefore also the product must be cooled with ice. After homogenization, the product was stored in a container filled with ice for at least 30 minutes.

### Synthesis VI

For the synthesis of LENOX in hydroxyethyl starch (HES, 6%), 1 g of bovine serum albumin (BSA) was dissolved in 20 mL of HES (6%). First 4 mL PFD and then 0.4 g lecithin were added to this solution. The mixture was pre-emulsified with an Ultra-Turrax (9,500 rpm). The pre-emulsion was then added to the high-pressure homogenizer and the entire volume was processed seven times at a pressure of 20,000 PSI (approx. 1379 bar). During the process, the outlet coil of the device and therefore also the product must be cooled with ice. After homogenization, the product was stored in a container filled with ice for at least 30 minutes.

### Synthesis VII

For the synthesis of LENOX in sodium chloride (NaCl, 0.9%), 1 g of bovine serum albumin (BSA) was dissolved in 20 mL of NaCl (0.9%). First 4 mL PFD and then 0.4 g lecithin were added to this solution. The mixture was pre-emulsified with an Ultra-Turrax (9,500 rpm). The pre-emulsion was then added to the high-pressure homogenizer and at a pressure of 20,000 PSI (approx. 1379 bar) the entire volume was cycled through the high-pressure homogenizer seven times. During the process, the outlet coil of the device must be cooled and therefore the product must also be cooled with ice. After homogenization, the product was stored in a container filled with ice for at least 30 minutes.

### Synthesis VIII

For the synthesis of LENOX in Ringer's solution, 1 g of bovine serum albumin (BSA) was dissolved in 20 mL of Ringer's solution. First 4 mL PFD and then 0.4 g lecithin were added to this solution. The mixture was pre-emulsified with an Ultra-Turrax (9,500 rpm). The pre-emulsion was then added to the high-pressure homogenizer and at a pressure of 20,000 PSI (approx. 1379 bar) the entire volume was cycled through the high-pressure homogenizer seven times. During the process, the outlet coil of the device and therefore also the product must be cooled with ice. After homogenization, the product was stored in a container filled with ice for at least 30 minutes.

### Synthesis IX

For the synthesis of LENOX in STEEN Solution, 4 mL PFD and then 0.4 g lecithin were first added to 20 mL STEEN Solution. The mixture was pre-emulsified with an Ultra-Turrax (9,500 rpm). The pre-emulsion was then added to the high-pressure homogenizer and at a pressure of 20,000 PSI (approx. 1379 bar) the entire volume was cycled through the high-pressure homogenizer seven times. During the process, the outlet coil of the device and therefore also the product must be cooled with ice. After homogenization, the product was stored in a container filled with ice for at least 30 minutes.

### Synthesis X

For the synthesis of LENOX in Sterofundin ISO, 1 g of bovine serum albumin (BSA) was dissolved in 20 mL of Sterofundin ISO. First 4 mL PFD and then 0.4 g lecithin were added to this solution. The mixture was pre-emulsified using an Ultra-Turrax (9,500 rpm). The pre-emulsion was then added to the high-pressure homogenizer and at a pressure of 20,000 PSI (approx. 1379 bar) the entire volume was cycled through the high-pressure homogenizer seven times. During the process, the outlet coil of the device and thus also the product must be cooled with ice. After homogenization, the product was stored in a container filled with ice for at least 30 minutes.

### Basic Data of the Syntheses

The following data refer to an undiluted synthesis as it results from the high-pressure homogenizer.

### Synthesis V

### Particle Size Distribution:

In the following table, d stands for the hydrodynamic diameter of the particles and S for the range of the particle size distribution. d10 means that 10% of the measured particles are exactly the same size or smaller than the specified particle diameter, d50 corresponds to 50% and d90 to 90%. d10 75 nm therefore means that 10% of the measured particles were 75 nm or smaller.

| d10 [nm] | d50 [nm] | d90 [nm] | S |
|---|---|---|---|
| 80 ± 10 | 139 ± 12 | 204 ± 25 | 0.9 ± 0.1 |

Zeta potential: −72.9±0.7 mV
viscosity (at 200 s⁻¹):
20 °C: 1.85±0.10 mPa·s (n=6)
37 °C: 1.21±0.07 mPa·s (n=6)
Oxygen release: 309±94 hPa (n=6)

### Long-Term Stability

LENOX was stored upright at 4 °C in completely filled 2 mL reaction vessels. The emulsion was analyzed every 7 days over a period of 42 days. Each batch of LENOX was divided into six reaction vessels so that a closed vessel was used each day and the already opened vessels were discarded.

### Viscosity:

| Time [d] | Viscosity (20 °C) at 200 s⁻¹ [mPas] |
|---|---|
| 0 | 1.68 ± 0.29 |
| 7 | 1.78 ± 0.20 |
| 14 | 1.80 ± 0.27 |
| 21 | 1.78 ± 0.25 |
| 28 | 1.69 ± 0.25 |
| 35 | 1.77 ± 0.22 |
| 42 | 1.59 ± 0.30 |

### Particle Size Distribution:

| Time [d] | d10 [nm] | d50 [nm] | d90 [nm] | S |
|---|---|---|---|---|
| 0 | 80 ± 10 | 139 ± 12 | 204 ± 25 | 0.9 ± 0.1 |
| 7 | 148 ± 27 | 234 ± 38 | 323 ± 51 | 0.7 ± 0.1 |
| 14 | 168 ± 24 | 256 ± 35 | 346 ± 47 | 0.7 ± 0.0 |
| 21 | 167 ± 26 | 255 ± 30 | 347 ± 36 | 0.7 ± 0.1 |
| 28 | 162 ± 26 | 259 ± 36 | 361 ± 48 | 0.8 ± 0.1 |
| 35 | 168 ± 36 | 264 ± 42 | 364 ± 51 | 0.7 ± 0.1 |
| 42 | 160 ± 29 | 258 ± 34 | 363 ± 40 | 0.8 ± 0.1 |

### Oxygen Release

Day 0: 309±94 hPa
Day 42: 379±75 hPa

### LENOX in Various Perfusion Solutions

The compatibility of LENOX with clinically relevant crystalloid and colloidal solutions is essential for a possible application. Colloidal volume replacement solutions are only used in exceptional clinical situations, but are widely used in preclinical research. For this reason, LENOX was synthesized and analyzed in various solutions.

### Synthesis VI, Synthesis VII, Synthesis VIII, Synthesis IX and Synthesis X

### Particle Size

| | d10 [nm] | d50 [nm] | d90 [nm] | S |
|---|---|---|---|---|
| HES 6% | 88 ± 4 | 175 ± 1 | 271 ± 5 | 1.0 ± 0.1 |
| NaCl 0.9% | 71 ± 4 | 152 ± 3 | 239 ± 14 | 1.1 ± 0.1 |
| Ringer solution | 73 ± 3 | 156 ± 2 | 246 ± 8 | 1.1 ± 0.1 |
| STEEN | 45 ± 1 | 204 ± 6 | 525 ± 28 | 2.4 ± 0.1 |
| Solution | | | | |
| Sterofundin | 89 ± 5 | 164 ± 3 | 243 ± 11 | 0.9 ± 0.1 |
| ISO | | | | |

Viscosity (at 200 s⁻¹)

| | Viscosity (20 °C) | Viscosity (20 °C) |
|---|---|---|
| | [mPas] | [mPas] |
| HES 6% | 3.19 ± 0.03 | 2.04 ± 0.02 |
| NaCl 0.9% | 1.84 ± 0.06 | 1.23 ± 0.07 |
| Ringer solution | 1.76 ± 0.03 | 1.13 ± 0.02 |
| STEEN Solution | 4.45 ± 0.18 | 3.05 ± 0.14 |
| Sterofundin ISO | 1.71 ± 0.06 | 1.12 ± 0.04 |

### Oxygen Release:

| | | | STEEN | |
|---|---|---|---|---|
| HES 6% | NaCl 0.9% | Ringer solution | Solution | Sterofundin ISO |
| 392 ± 48 hPa | 384 ± 86 hPa | 359 ± 58 hPa | 392 ± 62 hPa | 362 ± 51 hPa |

### LITERATURE

Information for healthcare professionals Konakion, status of information, January 2015, 26.07.2004
Information for healthcare professionals Albunorm, status of information, July 2018, approval number PEI.H.04333.02.1
Hoogevest P, Tiemessen H, Metselaar J M, Drescehr S, Fahr A. The Use of Phospholipids to Make Pharmaceutical Form Line Extensions. European Journal of Lipid Sciences and Technology. (2021): 123 (4), 2000297
Riess J. Oxygen Carriers (“Blood Substitutes”) Raison d'Etre, Chemistry, and Some Physiology. Chem. rev. (2001): 101, 2797-2919
Riess J. Understanding the Fundamentals of Perfluorocarbons and Perfluorocarbon Emulsions Relevant to In Vivo Oxygen Delivery. Artificial Cells, Blood Substitutes, and Biotechnology (2005), 33: 47-63, 2005
Jahr J. Blood Substitutes and Oxygen Therapeutics: A Review. Anesth Analg. (2021): 132(1):119-129. doi: 10.1213/ANE.0000000000003957.
Lambert E, Janjic J M. Quality by design approach identifies critical parameters driving oxygen delivery performance in vitro for perfluorocarbon based artificial oxygen carriers. Sci Rep (2021) 2021 Mar. 10; 11(1):5569. doi: 10.1038/s41598-021-84076-1.
Jaegers J, Haferkamp S, Arnolds O, Moog D, Wrobeln A, Nocke F, Cantore M, Putz S, Hartwig A, Franzkoch R, Psathaki O E, Jastrow H, Schauerte C, Stoll R, Kirsch M, Ferenz K B. Deciphering the Emulsification Process to Create an Albumin-Perfluorocarbon-(o/w) Nanoemulsion with High Shelf Life and Bioresistivity. Langmuir. 2022 Aug. 30; 38(34):10351-10361. doi: 10.1021/acs.langmuir.1c03388. Epub 2022 Aug. 15. PMID: 35969658.
Jagers J, Kirsch M, Cantore M, Karaman O and Ferenz K B. Artificial oxygen carriers in organ preservation: Dose dependency in a rat model of ex-vivo normothermic kidney perfusion. Artif Organs (2022) Apr. 18. doi: 10.1111/aor.14264.
Artif Organs. 2022 September; 46(9):1783-1793. doi: 10.1111/aor.14264. Epub 2022 Apr. 23. PMID: 35435266
Jägers J, Wrobeln A and Ferenz K B. Perfluorocarbon based oxygen carriers: From Physics to Physiology. Pflugers Arch (2021) 473 (2): 139-150, doi: 10.1007/s00424-020-02482-2
Wrobeln A, Jägers J, Quinting T, Schreiber T, Kirsch M, Fandrey J and Ferenz K B. Albumin-derived Perfluorocarbon-based Artificial Oxygen Carriers Can Avoid Hypoxic Tissue Damage in Massive Hemodilution. Sci rep (2020) 10 (1):11950. doi: 10.1038/s41598-020-68701-z.
Mayer, D, Guerrero F, Goanvec C, Hetzel L, Linders J, Ljubkovic M, Kreczy A, Mayer C, Kirsch M and Ferenz K B. Prevention of Decompression Sickness by Novel Artificial Oxygen Carriers. Med Sci Sports Exerc (2020) 52(10): 2127-2135, doi: 10.1249/MSS.0000000000002354.
Ferenz K B and Steinbicker A. Artificial oxygen carriers—past, present and future—a review of the most innovative and clinically relevant concepts. J Pharmacol Exp Ther (2019) 369: 300-10, doi: 10.1124/jpet.118.254664.
Wrobeln A, Schlüter K D, Linders J, Mayer C and Ferenz K B. Functionality of albumin-derived perfluorocarbon-based artificial oxygen carriers in the Langendorff-heart. Artif Cells Nanomed Biotechnol (2017) 45(4): 723-30, doi: 10.1080/21691401.2017.1284858.
Wrobeln A, Laudien J, Groß-Heitfeld C, Linders J, Mayer C, Wilde B, Knoll T, Naglav D, Kirsch M and Ferenz K B (2017). Albumin-derived perfluorocarbon-based artificial oxygen carriers: A physico-chemical characterization and first in vivo evaluation of biocompatibility. Eur J Pharm Biopharm (2017) 115: 52-64, doi: 10.1016/j.ejpb.2017.02.015.
Freire M G, Dias A M A, Coelho M A Z, Coutinho J A P, Marrucho I M. Aging mechanisms of perfluorocarbon emulsions using image analysis. Journal of Colloid and Interface Science. Volume 286. Issue 1. 2005. pages 224-232, doi: https://doi.org/10.1016/j.jcis.2004.12.036
Arnolds O, Cantore M, Ferenz K B, Haferkamp S, Jagers J, Kirsch M, Metzger J, Nocke F, Putz S, Schauerte C, Stoll R. Composition containing artificial oxygen carriers for the prevention of organ damage in transplant organs. Patentanmeldung DE102021211272.2
Deuchar G A, Brennan D, Holmes W M, Shaw M, Macrae I M, Santosh C. Perfluorocarbon Enhanced Glasgow Oxygen Level Dependent (GOLD) Magnetic Resonance Metabolic Imaging Identifies the Penumbra Following Acute Ischemic Stroke. Theranostics. 2018 Feb. 12; 8(6):1706-1722. doi: 10.7150/thno.21685

1. Method for the production of artificial oxygen carriers based on perfluorocarbons (PFOCs), comprising:
preparing an aqueous albumin solution comprising albumin and mixing the aqueous albumin solution with at least one artificial oxygen carrier based on perfluorocarbons;
adding lecithin;
performing rapid stirring under cooling to initiate pre-emulsification of the aqueous albumin solution and artificial oxygen carrier as a pre-emulsion;
emulsifying the pre-emulsion as an emulsion under pressure via a high-pressure homogenizer under cooling; and
storing the emulsion for at least 30 min under cooling.

2. Method according to claim 1, wherein the albumin is suitably dissolved in an aqueous buffer, water, and a balanced whole electrolyte solution for infusion therapy, wherein the electrolyte solution comprises at least one of Sterofundin® ISO, Ringerfundin®, STEEN-Solution, OCS solution, Ringer's solution or hydroxy-ethyl starch.

3. Method according to claim 1, wherein the artificial oxygen carrier based on perfluorocarbons is selected from the group consisting of perfluorodecalin, perfluorodecylbromide, perfluorotributylamine, perfluoromethylcyclohexylpiperidine, perfluorodichloroctane, perfluorotertbutylcyclohexane, perfluoro-15-crown-5-ether, dodecafluoropentane, perfluorooctylbromide and perfluorocyclohexane.

4. Method according to claim 1, wherein the albumin is selected from the group consisting of mammalian albumin, human serum albumin (HSA) and bovine serum albumin (BSA), or derivatives thereof.

5. Method according to claim 1, wherein the pre-emulsification is carried out by rapid stirring with a homogenizer between 8000 and 12000 rpm.

6. Method according to claim 1, wherein the emulsification of the pre-emulsion takes place at a pressure between 10,000 PSI (approx. 689 bar) and 40,000 PSI (approx. 2758 bar).

7. Method according to claim 1, wherein the cooling during at least one of the rapid stirring, emulsifying, and storing is carried out via at least one of at below 4 °C and on ice.

8. Method according to claim 1, wherein albumin is emulsified in water in a concentration between 2-20% perfluorodecalin 4 (PFD) in a concentration of between 10-50% and lecithin in a concentration of between 1-16%.

9. Method according to claim 1, wherein the emulsion is prepared at 20,000 PSI with bovine serum albumin in water and a particle diameter in the emulsion is between 50 nm and 400 nm.

10. Method according to claim 9, wherein a mean particle diameter with the bovine serum albumin is 92.5 nm±8.5 nm and the oxygen release is 3.99 μmol/mL±0.20 μmol/mL

11. Method according to claim 1, wherein a stable artificial oxygen carrier is produced exclusively from the aqueous albumin solution, the artificial oxygen carrier based on perfluorocarbons, and the lecithin.

12. A stable artificial oxygen carrier prepared by a process according to claim 1.

13. Method according to claim 12, wherein the stable artificial oxygen carrier consists of 5% albumin, perfluorodecalin (PFD) in a concentration of 17% and lecithin in a concentration of 2% in a medium selected from the following media: water, Sterofundin® ISO, Ringerfundin®, STEEN-Solution, OCS solution, Ringer solution or hydroxy ethyl starch.

14. Method according to claim 12, wherein the stable artificial oxygen carrier has a higher stability compared to non-lecithin-containing artificial oxygen carriers based on perfluorocarbons (PFOCs) due to an increased viscosity of the stable artificial oxygen carrier.

15. Method according to claim 1, wherein the emulsion comprises a synthetic blood substitute.

16. Method according to claim 1, wherein the artificial oxygen carrier is used as a perfusion or transfusion solution or as a synthetic blood substitute.

17. Method according to claim 6, wherein emulsification of the pre-emulsion takes place at a pressure 30,000 PSI (2068 bar) and is repeated one or more times.
